(19) United States
(12) Patent Application Publication
(10) Pub. No.: US 2024/0041955 A1
(43) Pub. Date: Feb. 8, 2024

(54) COSMETIC, NUTRACEUTICAL OR DERMATOLOGICAL USE OF A LACTOBACILLUS CRISPATUS STRAIN AND/OR OF A COMPOSITION COMPRISING SAME

(71) Applicants: BASF Beauty Care Soutions France SAS, Lyon (FR); BASF Corporation, Florham Park, NJ (US)

(72) Inventors: Neethu ABRAHAM, Ardsley, NY (US); Valérie ANDRE, Lyon (FR); Nicolas Stefano DEL BENE, Florham Park, NJ (US); Allison GARLET, Tarrytown (US); Manon GAULT, Lyon (FR); Sabrina LEOTY-OKOMBI, Lyon (FR)

(21) Appl. No.: 18/265,694

(22) PCT Filed: Dec. 14, 2021
(86) PCT No.: PCT/FR2021/052325

§ 371 Date: Jun. 7, 2023

(30) Foreign Application Priority Data
Dec. 15, 2020 (FR) FR2013222

(51) Int. Cl. A61K 35/747; A61K 8/99; A61Q 19/08; A61P 17/00

(52) U.S. Cl. CPC A61K 35/747; A61K 8/99; A61Q 19/08; A61P 17/00

(57) ABSTRACT

The present invention relates to the cosmetic, nutraceutical and/or pharmaceutical, notably dermatological, use of a strain of Lactobacillus crispatus and/or of a composition comprising it for maintaining and/or increasing the expression of extracellular matrix molecules of the skin and/or of mucous membranes and notably for maintaining and/or increasing the biomechanical properties of healthy skin and/or healthy mucous membranes, preferentially the firmness and/or elasticity and/or density of healthy skin and/or healthy mucous membranes.

The present invention relates to the cosmetic, nutraceutical and/or pharmaceutical, notably dermatological, use of a strain of Lactobacillus crispatus and/or a composition comprising it for maintaining and/or increasing the expression of extracellular matrix molecules of the skin and/or of the mucous membranes and notably for maintaining and/or increasing the biomechanical properties of healthy skin and/or healthy mucous membranes, preferentially the firmness and/or elasticity and/or density of healthy skin and/or healthy mucous membranes, even more preferentially the firmness and/or elasticity of healthy skin and/or healthy mucous membranes.

The structure and properties of the skin change as a result of complex biological, physical and biomechanical processes, leading to a loss of elasticity, firmness and density of the dermis, notably as a result of a change in the molecules of the extracellular matrix.

The extracellular matrix of the skin and mucous membranes, known as the ECM, is composed of a combination of three types of molecules:
fibers: collagen and elastin,
glycoproteins: less abundant than fibers but have a more important role in cell adhesion, such as fibronectin and laminin,
highly hydrated polysaccharides constituting a gel filling the matrix. This matrix plays a predominant role in maintaining the structure and properties of the skin, notably the elasticity, firmness and density of the dermis.

Since these ECM molecules are notably synthesized by fibroblasts and keratinocytes, renewal of said cells is essential for maintaining the skin's biomechanical properties. Elastin is a protein that is a component of elastic fibers by combining with other molecules such as fibrillins and MAGP (Microfibrillar Associated Glycoproteins).

Elastin is synthesized in the form of soluble tropoelastin which acquires its physicochemical properties (insolubility, elasticity) after its intra- and intermolecular crosslinking by means of a lysyl oxidase (LOX) and its deposition on the microfibrils. In the skin, functional elastic fibers are formed by the dermal fibroblasts, but some components necessary for producing elastic fibers have also been found in epidermal cells.

The turnover rate of elastic fibers is very low in adult life, although the overall elastin content of the skin may increase. In the newborn, the microfibrils are not all completely covered with elastin, and become so around the age of puberty. From the age of 40, inclusions appear on the fibers, more frequent in women, followed by fragmentation of the elastic fibers and their disappearance under the dermoepidermal junction (DEJ). This fragmentation and/or disappearance under the DEJ are reflected by a decrease in skin elasticity and the formation of wrinkles. The synthesis of nonfunctional elastic fibers is observed during photoaging, but this increase is accompanied by an accelerated decrease in elastic fibers under the DEJ.

Collagen is a constituent protein of the extracellular matrix (ECM), present in large amounts in vertebrate tissues. It is a large family comprising 29 different types.

Among the various types of collagen are, in particular, type I collagen in many human tissues such as tendons, ligaments, cornea and skin, more precisely located in the dermis. Type I collagen is the major collagen of the skin.

Fibrillar collagen is formed from procollagen fibers, and then assembled in a network and stabilized by crosslinking. It is collagen that gives tissues their mechanical strength, and as a result helps to maintain their firmness.

During aging of the skin, the rate of synthesized collagen decreases overall and its degradation increases, which leads to a decrease in firmness. This phenomenon is even more pronounced when the skin is subjected, inter alia, to UV rays. This is referred to as photobiological aging.

The molecules of the extracellular matrix are thus the subject of numerous studies and innovations in the field of cosmetics for the development and improvement of cosmetic active ingredients directed toward maintaining the biomechanical properties of the skin.

In recent years, advanced genome sequencing techniques have revealed that millions of microorganisms (bacteria, viruses, fungi) live on the skin and make up the cutaneous microbiota, also known as the skin flora. Most of these microorganisms (this microbiota) are commensal and beneficial to the skin. They cohabit and interact with skin cells and some have developed symbiosis with the skin. They participate in defending the skin by synthesizing or stimulating the synthesis of skin defense molecules. They participate in repairing the skin in the event of injury and fight against inflammation. These beneficial commensal microorganisms secrete molecules and metabolites which participate in the homeostasis and balance of the skin.

The composition and quality of the skin's microbiota changes with aging.

Surprisingly, the Applicant has demonstrated that, with age, certain bacteria are less present and less abundant on the skin. This is notably the case for the bacterium Lactobacillus crispatus.

Most of the solutions existing on the market deal with the visible consequences of aging and do not take into account the cutaneous microbiota, some species of which live in symbiosis with the skin cells and participate in its balance and appearance.

The present invention has the advantage of being more complete in the sense that it rebalances the microbiota of aged skin by supplementing it with bacteria of the cutaneous flora which diminish over time. These bacteria in turn contribute toward the rejuvenation and long-lasting protection of the skin against external attack, by reactivating biological processes which diminish with age.

Probiotic bacteria and derivatives thereof are already used for treating skin aging problems, but in most cases the bacteria used do not come from the skin. The bacterium according to the present invention has the advantage of being naturally present on young skin.

Patent application WO 2009/031106 discloses the use of a combination of hesperidin and a probiotic for preventing the reduction of or reinforcing the skin's barrier function. Said application does not disclose the use of a Lactobacillus crispatus strain for maintaining and/or increasing the expression of skin and/or mucous membrane extracellular matrix molecules.

Patent application WO 2019/111189 discloses the use comprising a probiotic of the genus Lactobacillus for preventing UV radiation-induced damage. Said application does not disclose the use of a Lactobacillus crispatus strain for maintaining and/or increasing the expression of skin and/or mucous membrane extracellular matrix molecules.

Thus, to the Applicant's knowledge, no prior art discloses or suggests the cosmetic, nutraceutical and/or pharmaceutical use, notably the dermatological use, of a strain of Lactobacillus crispatus and/or a composition comprising it for maintaining and/or increasing the expression of skin and/or mucous membrane extracellular matrix molecules, nor for maintaining and/or increasing the biomechanical properties of the skin and/or mucous membranes, nor for maintaining and/or increasing the expression of skin and/or mucous membrane collagens and/or skin and/or mucous membrane elastic fibers.

A first subject of the present invention is the cosmetic and/or nutraceutical use of a strain of Lactobacillus crispatus for maintaining and/or increasing the expression of extracellular matrix molecules of healthy skin and/or healthy mucous membranes.

A subject of the invention is also a cosmetic care process, characterized in that it comprises the topical application to at least one area of healthy skin and/or healthy mucous membranes of a strain of Lactobacillus crispatus according to the invention, or of a cosmetic composition comprising it, for maintaining and/or increasing the expression of extracellular matrix molecules of healthy skin and/or healthy mucous membranes, preferably for maintaining and/or increasing the expression of collagens of healthy skin and/or healthy mucous membranes and/or elastic fibers of healthy skin and/or healthy mucous membranes, and/or for maintaining and/or increasing the biomechanical properties of healthy skin and/or healthy mucous membranes, notably the firmness and/or elasticity and/or density, preferably the firmness.

A subject of the invention is also a strain of Lactobacillus crispatus according to the invention, or a dermatological composition comprising it, for treating and/or preventing pathologies of the skin and/or mucous membranes involving a decrease in the amount and/or expression of extracellular matrix molecules of the skin and/or mucous membranes, notably a decrease in the amount and/or expression of skin and/or mucous membrane collagens and/or skin and/or mucous membrane elastic fibers, and/or a decrease in the biomechanical properties of the skin and/or mucous membranes, notably a decrease in the firmness of the skin and/or mucous membranes and/or in the elasticity and/or density.

The invention also relates to a strain of Lactobacillus crispatus deposited under the Budapest Treaty at the Pasteur Institute (28 rue du Docteur Roux, F-75024 Paris cedex 15) on 09/09/2020 under the designation CNCM I-5579 or a cosmetic composition comprising it, for maintaining and/or increasing the expression of extracellular matrix molecules of healthy skin and/or healthy mucous membranes, notably for maintaining and/or increasing the expression of collagens of healthy skin and/or healthy mucous membranes and/or elastic fibers of healthy skin and/or healthy mucous membranes, and/or for maintaining and/or increasing the biomechanical properties of healthy skin and/or healthy mucous membranes, notably the firmness and/or elasticity and/or density, preferentially the firmness.

Finally, the invention relates to a strain of Lactobacillus crispatus deposited under the Budapest Treaty at the Pasteur Institute (28 rue du Docteur Roux, F-75024 Paris cedex 15) on 09/09/2020 under the designation CNCM I-5579 or a dermatological composition comprising it, for treating and/or preventing skin and/or mucous membrane pathologies involving a decrease in the amount and/or expression of extracellular matrix molecules of the skin and/or mucous membranes, notably a decrease in the amount and/or expression of skin and/or mucous membrane collagens and/or elastic fibers, and/or a decrease in the amount and/or biomechanical properties of the skin and/or mucous membranes, notably a decrease in the firmness of the skin and/or mucous membranes, and/or a decrease in the elasticity of the skin and/or mucous membranes, and/or a decrease in the density of the skin and/or mucous membranes.

For the purposes of the present invention, the term “cosmetic use and/or composition” means a non-pharmaceutical use and/or composition, i.e. one that is not intended for therapeutic use and that is applied to a “healthy” part of the body, in particular to a “healthy” area of the skin and/or mucous membranes.

For the purposes of the present invention, the expression “nutraceutical use and/or nutraceutical composition” means a use and/or a composition for non-pharmaceutical oral administration, i.e. one which does not need a therapeutic treatment.

For the purposes of the present invention, the term “healthy skin” or “healthy mucous membranes” means an area of skin or mucous membranes to which the strain according to the invention is applied and which is said by a dermatologist to be “nonpathological”, i.e. free of infection, scarring, disease or skin disorder such as candidiasis, impetigo, psoriasis, eczema, acne or dermatitis, or wounds or injuries.

According to the invention, “mucous membrane(s)” means the ocular mucous membrane, the nasal mucous membrane, the auricular mucous membrane, the urogenital mucous membrane and/or the oral mucous membrane, notably the oral, labial and/or gingival mucous membrane, preferentially the ocular and/or oral mucous membrane, and more preferentially, the labial and/or ocular mucous membrane. Advantageously, said term does not include the vaginal mucous membrane.

For the purposes of the present invention, the term “maintaining and/or increasing the expression of extracellular matrix molecules” means preventing a decrease in and/or increasing the level of gene expression, i.e. mRNAs (messenger RNAs), and/or protein synthesis of the extracellular matrix molecules of healthy skin and/or healthy mucous membranes, treated with the strain according to the invention, relative to the level of gene expression and/or protein synthesis detected in the absence of the strain according to the invention. Preferentially, it is a matter of maintaining and/or increasing the protein expression of extracellular matrix molecules.

According to the invention, the term “ECM molecules” means organic molecules which constitute and/or are contained in the extracellular matrix and which are synthesized by cells, of human or animal origin.

These are notably the constituent proteins of the ECM, in particular selected from:
the family of ECM collagens,
the family of ECM elastic fibers: elastin, tropoelastin, elastin-associated proteins, notably fibrillin 1, lysyl oxidases, notably LOX and LOXL, EBP (Elastin Binding Protein), fibullins 3 and 5, emilin 1 and 2.

These are notably the constituent proteoglycans of the ECM, in particular the secreted proteoglycans chosen from perlecan, versican, leucine-rich proteoglycans (SLRPs) notably decorin, biglycan and lumican.

It also includes the constituent glycoproteins of the ECM, notably fibronectin, laminin, SPARC (Secreted Protein Rich in Cystein) and tenascin.

It also includes the glycosaminoglycans (GAG) constituting the ECM, in particular hyaluronic acid, heparan sulfate, dermatan sulfate and chondroitin sulfate.

It also includes ECM growth factors which are proteins contained in the ECM, notably VEGF, PDGF, HGF, and FGFs, in particular FGF2 and FGF 7.

The term “ECM molecules” refers to molecules in the ECM or in other compartments, notably intracellular and extracellular compartments, in the culture medium, possibly in the form of precursors.

According to the invention, the term “ECM molecule precursor” means a native and/or intermediate form of the ECM molecule, synthesized by the cell, before constituting or being contained in the ECM. These forms undergo maturation phenomena before being in the ECM and are thus often designated by the prefixes “Pro-” or “Tropo-”. They are, for example, procollagens, tropocollagens, proelastin and tropoelastin.

Preferentially, the extracellular matrix molecules are chosen from the collagens of healthy skin and/or healthy mucous membranes, and/or the elastic fibers of healthy skin and/or healthy mucous membranes.

For the purposes of the present invention, the term “maintaining and/or increasing the expression of collagens of healthy skin and/or healthy mucous membranes and/or elastic fibers of healthy skin and/or healthy mucous membranes” means preventing a decrease in and/or increasing the level of gene expression, i.e. the expression of mRNAs (messenger RNAs), and/or protein synthesis of collagens of healthy skin and/or healthy mucous membranes and/or elastic fibers of healthy skin and/or healthy mucous membranes treated with the strain according to the invention, relative to the gene expression and/or protein synthesis measured in the absence of the strain according to the invention.

In particular, it is an increase in the level of gene expression and/or protein synthesis of collagens and/or elastic fibers of at least 20%, preferentially of at least 40% and even more preferentially of at least 50%, relative to the level of gene and/or protein expression of collagens and/or elastic fibers measured in the absence of the strain of Lactobacillus crispatus according to the invention.

In a preferential mode of the invention, it is a matter of increasing the level of protein expression of fibulin-5 and/or emilin-1 by at least 20%, preferentially by at least 40% and even more preferentially by at least 50% relative to the level of protein expression measured in dermal fibroblast cells cultured in vitro in the absence of the strain, as described, for example, in Example 3.

Advantageously, the measurement of the increase in protein expression of fibulin-5 and/or emilin-1 is performed by in vitro measurement, preferentially after analysis by capillary electrophoresis, for example according to the method presented in Example 3.

For the purposes of the present invention, the term “analysis by capillary electrophoresis” means the technique consisting in attaching an antibody specific to the protein studied, fibulin-5 and/or emilin-1, to said protein, conjugated to a peroxidase and a chemiluminescent substrate, for the purpose of detecting the protein by measuring the chemiluminescent signal.

For the purposes of the present invention, the term “collagen” means collagen proteins of type I, III, IV, V, VI, VII, XII, XIII, XIV, XVI, XVII, XXIV, XXIX, present in healthy skin and/or healthy mucous membranes. According to an advantageous mode, the collagens are chosen from type I, Ill, IV, V and/or XIV collagens, preferentially type I, IV and/or V collagens, more preferentially type I collagen.

For the purposes of the present invention, the term “type I collagen protein” means the collagen protein present in the skin, cornea, tendons, ligaments and bones, more preferentially in the skin. Thus, in a preferential mode of the invention, the strain of Lactobacillus crispatus according to the invention is thus used for maintaining and/or increasing the protein expression of type I collagen in healthy skin and/or healthy mucous membranes.

According to an advantageous mode of the invention, this is an increase relative to the level of gene and/or protein expression of collagen measured in dermal fibroblast cells cultured in vitro in the absence of the strain of Lactobacillus crispatus according to the invention.

In a preferential mode of the invention, this is an increase in the level of protein expression of type I collagen of at least 10%, preferentially of at least 50% and even more preferentially of at least 20% relative to the level of protein expression measured in dermal fibroblast cells cultured in vitro in the absence of the strain according to the protocol as described, for example, in Example 2.

Advantageously, the measurement of the increase in protein expression is performed by in vitro measurement, preferentially after enzyme-linked immunosorbent assay or ELISA, for example according to the method as presented in Example 2.

For the purposes of the present invention, the term “enzyme immunoassay” means the technique consisting in fixing an antibody specific to a given collagen protein, preferentially type I collagen protein, onto said protein, for the purpose of revelation by fluorescence measurement. Advantageously, the revelation is done by fluorescence.

According to another advantageous mode, the measurement of the increase in gene expression is performed by in vitro measurement, preferentially after RT-qPCR.

In a preferred embodiment, the strain according to the invention is used for maintaining and/or increasing the biomechanical properties of healthy skin and/or healthy mucous membranes, preferentially the firmness and/or elasticity and/or density of healthy skin and/or healthy mucous membranes, more preferentially the firmness of healthy skin and/or mucous membranes, in particular of healthy dermis and/or the elasticity of healthy skin and/or mucous membranes, in particular of healthy dermis.

For the purposes of the present invention, the term “biomechanical properties of healthy skin and/or healthy mucous membranes” means the firmness and/or elasticity and/or density and/or resistance to compression and/or resistance to stretching and/or flexibility and/or stretchability and/or the ability to withstand deformation of healthy skin and/or healthy mucous membranes. Preferentially it is the firmness and/or elasticity and/or density of healthy skin and/or healthy mucous membranes, in particular of healthy dermis, even more preferentially it is the firmness of healthy skin and/or healthy mucous membranes, in particular of healthy dermis and/or the elasticity of healthy skin and/or healthy mucous membranes, in particular of healthy dermis.

The biomechanical properties of healthy skin and/or healthy mucous membranes may be studied by measurement techniques known to those skilled in the art, notably by pulling, twisting, suction, indentation, levatometry, ballistometry, by high-resolution ultrasound imaging or elastography, preferentially by indentation or by high-resolution ultrasound imaging.

For the purposes of the present invention, the term “maintaining and/or increasing the biomechanical properties of healthy skin and/or healthy mucous membranes” means preventing a decrease in and/or increasing the biomechanical properties of healthy skin and/or healthy mucous membranes, preferentially the firmness of healthy skin and/or healthy mucous membranes, in particular of healthy dermis, and/or the elasticity of healthy skin and/or healthy mucous membranes, in particular of healthy dermis, and/or the density of healthy skin and/or healthy mucous membranes, in particular of healthy dermis, treated with the strain according to the invention relative to the biomechanical properties of healthy skin and/or healthy mucous membranes measured in the absence of the strain according to the invention.

Preferentially, it is an increase of at least 5%, preferentially 10%, of the biomechanical properties of healthy skin and/or healthy mucous membranes treated with the strain of Lactobacillus crispatus according to the invention relative to the biomechanical properties of healthy skin and/or healthy mucous membranes measured in the absence of the strain of Lactobacillus crispatus according to the invention.

For the purposes of the present invention, from a cosmetic point of view, the term “maintaining and/or increasing the firmness of healthy skin and/or healthy mucous membranes, in particular of healthy dermis” means preventing the reduction in and/or increasing, for esthetic purposes, the firmness of healthy skin and/or healthy mucous membranes, notably those that have lost firmness notably as a result of intrinsic factors. The intrinsic factors may be tissue aging of the skin and/or mucous membranes, i.e. chrono-induced aging, which may be found, for example, in “mature” skin, i.e. the skin of people over 40 years old. It may be due to cellular stress, nonpathological physiological variations such as a change in diet, or hormonal variations, particularly during puberty, pregnancy, menopause and/or andropause. This decrease in firmness may also occur as a result of extrinsic factors, such as aggressive environmental agents, for instance pollution, smoke, tobacco, toxins, or climatic and/or mechanical attacking factors.

According to a preferential mode, the decrease in firmness is not UV radiation-induced.

Preferentially, it is an increase in the firmness of healthy skin and/or healthy mucous membranes, in particular of healthy dermis, of at least 1%, preferentially of at least 3%, more advantageously of at least 5% in the presence of a strain of Lactobacillus crispatus according to the invention. In one advantageous embodiment of the invention, it is an increase measured in vivo, preferentially on the skin of the human face.

For the purposes of the present invention, from a cosmetic point of view, the term “maintaining and/or increasing the elasticity of healthy skin and/or healthy mucous membranes, in particular of healthy dermis” means preventing the reduction in and/or increasing, for esthetic purposes, the elasticity of healthy skin and/or healthy mucous membranes which have lost elasticity notably as a result of intrinsic factors, such as tissue aging of the skin and/or mucous membranes, i.e. chrono-induced aging, which is found for example in “mature” skin, i.e. the skin of people over 40 years old, in particular over 50 years old, cellular stress, nonpathological physiological variations such as a change in diet, or hormonal variations, in particular during puberty, pregnancy, menopause, and/or andropause. This decrease in elasticity may also occur as a result of extrinsic factors, such as aggressive environmental agents, for instance pollution, smoke, tobacco, toxins, or climatic and/or mechanical attacking factors.

According to a preferential mode, the decrease in elasticity is not UV radiation-induced.

In addition, the term “increasing the elasticity” of the skin and/or mucous membranes means an increase in elasticity measured in vivo, of at least 2%, advantageously of at least 4%, more advantageously of at least 6% in the presence of the strain according to the invention relative to the elasticity detected in the absence of the strain. In a preferential embodiment of the invention, this measurement is taken on human facial skin. In a preferential mode, this increase is evaluated after at least 10, preferentially 28, more preferentially 30 and advantageously at least 56 days of treatment in the presence of the strain according to the invention.

More advantageously, the elasticity is evaluated by measuring its various elasticity components, notably net elasticity, elastic recovery and/or overall elasticity, notably as described in Example 4.

For the purposes of the present invention, from a cosmetic point of view, the term “maintaining and/or increasing the density of healthy skin and/or healthy mucous membranes, in particular of healthy dermis” means preventing the reduction in and/or increasing, for esthetic purposes, the density of healthy skin and/or healthy mucous membranes, notably those that have lost density notably as a result of intrinsic factors. The intrinsic factors may be tissue aging of the skin and/or mucous membranes, i.e. chrono-induced aging, which may be found, for example, in “mature” skin, i.e. the skin of people over 40 years old. It may be due to cellular stress, nonpathological physiological variations such as a change in diet, or hormonal variations, particularly during puberty, pregnancy, menopause and/or andropause. This decrease in density may also occur as a result of extrinsic factors, such as aggressive environmental agents, for instance pollution, smoke, tobacco, toxins, or climatic and/or mechanical attacking factors.

According to a preferential mode, the decrease in density is not UV radiation-induced.

Preferentially, it is an increase in the density of healthy skin and/or healthy mucous membranes, in particular of healthy dermis, of at least 1%, preferentially of at least 3%, more advantageously of at least 5% in the presence of a strain of Lactobacillus crispatus according to the invention. In an advantageous embodiment of the invention, this is an increase measured in vivo, preferentially on human facial skin.

The in vivo measurement of firmness and/or elasticity and/or density may be performed according to conventional methods known to those skilled in the art, notably by measurement using a cutometer, a Tonoderm™, an ultrasound scanner, or a DynaSKIN® associated with a dermaTOP.

According to the present invention, the Lactobacillus crispatus strain may be used in whole form, notably viable and/or inactivated, notably dead, and/or in the form of a lyzate, and/or in the form of one or more of its fractions, and/or in the form of one or more of its metabolites, preferentially its secretome.

For each of its forms, the strain according to the invention may be isolated or combined with its fermentation and/or culture medium.

Preferentially, the strain according to the invention is combined with its fermentation and/or culture medium.

For the purposes of the present invention, the term “isolated” means not mixed with one or more compounds that may be associated therewith in its fermentation and/or growth medium.

For the purposes of the present invention, the term “whole form” means its native form, a form in which the bacterial envelope is intact, as opposed to a lyzed form. When it is used in whole form, the Lactobacillus crispatus strain may be viable and/or inactivated and/or dead.

For the purposes of the present invention, the term “viable” refers to a Lactobacillus crispatus strain according to the present invention that is capable of forming colonies in culture.

The production of the strain of Lactobacillus crispatus in viable whole form may be performed according to any method conventionally known to those skilled in the art. Advantageously, it will be performed according to the protocol as described, for example, in Example 1.a or 1.f.

For the purposes of the invention, the term “inactivated” refers to a strain of Lactobacillus crispatus according to the present invention that is no longer capable, temporarily or definitively, of forming colonies in culture.

The inactivation of the strain of Lactobacillus crispatus may be performed via any method conventionally known to those skilled in the art. It may notably be inactivated by irradiation, heat treatment or, under certain conditions, by freeze-drying, by high-pressure treatment or by extrusion. Advantageously, it will be performed by heat treatment, more advantageously according to the protocol as described, for example, in Example 1.b.

Heat inactivation may be performed by incubating the Lactobacillus crispatus strain for a given period of time, advantageously from about 10 s to 90 min, preferentially from about 15 minutes to one hour, and at a temperature advantageously from about 60° C. to 150° C. Preferentially, it will be incubated for about 30 minutes at about 80° C.

For the purposes of the invention, the term “dead” refers to a strain of Lactobacillus crispatus according to the present invention that is no longer capable, definitively, of forming colonies in culture.

For the purposes of the invention, the term “lyzate” means material obtained following the destruction or dissolution of biological cells by means of a phenomenon known as cell lysis, thus causing the release of the intracellular biological constituents naturally contained in the cells of the microorganism under consideration and of fragments of cell membrane components. The lyzate used is thus formed from all the intracellular biological constituents and the constituents of the cell walls and membranes of the strain of Lactobacillus crispatus according to the invention.

The lyzate may be obtained via any method conventionally known to those skilled in the art. It may notably be obtained by an osmotic shock, a thermal shock, by ultrasound, by enzymatic lysis, by tyndallization, or under mechanical stress of the centrifugation type, or by increasing the pressure or combinations of these various technologies.

Preferentially, the lyzate will be obtained by a combination of mechanical action and pressure increase. More preferentially, the lyzate will be obtained according to the protocol as described, for example, in Example 1.b.

In a preferential embodiment of the invention, the lyzate will be combined with the bacterial culture and/or fermentation medium.

For the purposes of the present invention, the term “fractions” means a fragment of the Lactobacillus crispatus strain according to the present invention which is efficient in maintaining and/or increasing the expression of extracellular matrix molecules of healthy skin and/or healthy mucous membranes, and/or for maintaining and/or increasing the biomechanical properties of healthy skin and/or healthy mucous membranes. This fraction corresponds to a non-total portion of the intracellular biological constituents and cell wall and membrane constituents obtained by lysis of the Lactobacillus crispatus strain according to the invention.

According to a particular embodiment of the invention, the fractions may be the bacterial protoplasm.

In a preferential embodiment of the invention, the fractions will be combined with the bacterial culture and/or fermentation medium.

For the purposes of the present invention, the term “metabolites” means one or more organic and/or inorganic molecules derived from the metabolism of the Lactobacillus crispatus strain according to the present invention and which is also efficient in maintaining and/or increasing the expression of extracellular matrix molecules of healthy skin and/or healthy mucous membranes, and/or in maintaining and/or increasing the biomechanical properties of healthy skin and/or healthy mucous membranes. Preferentially, this is the secretome of the Lactobacillus crispatus strain according to the invention.

In a particular embodiment of the invention, the metabolites will be combined with the bacterial culture and/or fermentation medium.

For the purposes of the present invention, the term “secretome” means all of the organic and/or inorganic molecules secreted by the strain of Lactobacillus crispatus according to the present invention which is efficient in maintaining and/or increasing the expression of extracellular matrix molecules of healthy skin and/or healthy mucous membranes, and/or in maintaining and/or increasing the biomechanical properties of healthy skin and/or healthy mucous membranes.

The secretome may be obtained via any method conventionally known to those skilled in the art. Advantageously, it will be obtained according to the protocol as described, for example, in Example 1.d or 1e.

In a preferential embodiment of the invention, the secretome will be combined with the bacterial culture and/or fermentation medium.

According to a preferential embodiment of the invention, the Lactobacillus crispatus strain according to the invention will be used in viable whole form and/or in lyzate form.

The strain of Lactobacillus crispatus according to the invention may be derived from any known species of Lactobacillus crispatus. Preferentially, the strain of Lactobacillus crispatus is the species deposited under the Budapest Treaty at the Institut Pasteur (28 rue du Docteur Roux, F-75024 Paris cedex 15) on 09/09/2020 under the designation CNCM I-5579.

The advantage of this particular strain of Lactobacillus crispatus is that it is the first Lactobacillus crispatus species found naturally on healthy skin.

According to the invention, the strain of Lactobacillus crispatus according to the invention may be used alone, in the form of an active ingredient and/or in a cosmetic and/or nutraceutical and/or pharmaceutical composition, notably a dermatological composition, preferentially intended for topical and/or oral application, more preferentially for topical application.

When it is used in living whole form according to the invention, and when it is used alone, in the form of an active ingredient, the strain according to the invention may be in dry form, i.e. in powder form, advantageously in maltodextrin, preferably in a strain content of 10% to 80% (w/w) by weight of the strain, preferentially from 30% to 70% (w/w), more advantageously from 40% to 60% (w/w) by weight of the strain relative to the total weight of the powder.

Alternatively, the living whole form according to the invention may be used soluble and/or diluted in a solvent. Preferentially, this solvent contains less than 20% (v/v) by volume of water, more preferentially less than 5% (v/v) by volume of water; more preferentially, the solvent does not contain any water. Very preferentially, this solvent is a cosmetically acceptable oil.

When it is used in its whole inactivated form, notably dead, and/or in the form of a lyzate, and/or in the form of one or more of its fractions, and/or in the form of one or more of its metabolites, notably of its secretome, and when it is used alone in the form of an active ingredient, the strain according to the invention may be in dry form, i.e. in the form of a powder, advantageously in maltodextrin, preferentially in a strain content of from 5% to 80% (w/w) by weight of the strain, preferentially from 10% to 70% (w/w) by weight of the strain, very preferentially from about 20% to 50% (w/w) by weight of the strain relative to the total weight of powder, more advantageously from 10% to 50% (w/w) by weight of the strain relative to the total weight of the powder.

Alternatively, the inactivated whole forms, notably dead forms, and/or the lyzates, and/or the fractions, and/or the metabolites, notably the secretome according to the invention, may be used soluble and/or diluted in a solvent, notably a polar solvent, such as water, an alcohol, a polyol, a glycol such as pentylene glycol and/or hexylene glycol and/or caprylyl glycol, or a mixture thereof, preferentially a water-glycol or aqueous-alcoholic mixture, more preferentially comprising a glycol chosen from hexylene glycol, caprylyl glycol and mixtures thereof.

In another embodiment, the strain according to the invention may be incorporated into a cosmetic and/or nutraceutical composition comprising at least one cosmetically acceptable and/or nutraceutically acceptable excipient. Preferentially, it is incorporated into a cosmetic composition comprising at least one cosmetically acceptable excipient. For the purposes of the present invention, the term “cosmetically acceptable excipient” means a topically acceptable compound and/or solvent, i.e. one which does not induce an allergic response on contact with the skin, including the human scalp, and/or mucous membranes, and which is nontoxic and not chemically unstable.

In a preferential embodiment of the invention, the strain according to the invention is present in the cosmetic and/or dermatological composition in a content of between 1×10⁻⁴% and 10% (v/v) by volume, preferentially between 1×10⁻⁴% and 5% (v/v) by volume, more advantageously between 1×10⁻³% and 3% (v/v) by volume, more preferentially between 0.1% and 2% (v/v) by volume, relative to the total volume of the composition, said composition also comprising at least one cosmetically acceptable excipient.

Advantageously, the strain according to the invention is present in the cosmetic and/or dermatological composition, at a concentration of 1×10⁻⁴% to 10% by weight, preferentially between 1×10⁻⁴% and 5% by weight, and more advantageously between 1×10⁻³% and 0.5% by weight, relative to the total weight of the composition, said composition also comprising at least one cosmetically acceptable excipient.

In a particularly advantageous manner, when the strain according to the invention is used in the form of one or more of its metabolites, notably of its secretome, it is present in the cosmetic and/or dermatological composition at a concentration of from 0.05% to 0.5% by weight, more preferentially at a concentration of about 0.1% by weight, relative to the total weight of the composition, said composition also comprising at least one cosmetically acceptable excipient.

Particularly advantageously, when the strain according to the invention is used in viable whole form, it is present in the cosmetic and/or dermatological composition, at a concentration of from 0.01% to 0.1% by weight, more preferentially at a concentration of about 0.025% by weight, relative to the total weight of the composition, said composition also comprising at least one cosmetically acceptable excipient.

The cosmetic and/or dermatological composition of the invention may be chosen from an aqueous or oily solution, a cream or an aqueous gel or an oily gel, notably a shower gel, a shampoo; a milk; an emulsion, a microemulsion or a nanoemulsion, notably an oil-in-water or water-in-oil or multiple or silicon-based emulsion; a mask; a serum; a lotion; a liquid soap; a dermatological bar; an ointment; a balm; a butter; a mousse; a patch; an anhydrous product, which is preferably liquid, pasty or solid, for example in the form of makeup powders, wands or sticks, notably in the form of lipstick.

It may also be a makeup product or a makeup-removing product.

Advantageously, the strain of Lactobacillus crispatus or the composition of the invention is intended to be applied to all or part of the body and/or face and/or scalp, preferably the legs, thighs, arms, stomach, neckline, neck, armpits or lips, more preferentially all or part of the face, and preferentially the cheeks, forehead, chin, lips or contour of the eyes.

Advantageously, the strain and/or the composition comprising it is intended to be applied to an area of healthy skin showing sagging and/or a collapsed area of healthy skin and/or an area of healthy skin lacking in tone.

Thus, advantageously, the cosmetic composition is intended for topical application to healthy skin and/or healthy mucous membranes.

Preferentially, the strain of Lactobacillus crispatus according to the invention is particularly suitable for the formulation of a neutral and mild composition for the respect of the sebaceous gland, notably the skin including the scalp and/or the mucous membranes.

Alternatively, the strain of Lactobacillus crispatus according to the invention may be in any presentation form conventionally used for oral application, notably in the form of a nutraceutical active ingredient and/or a nutraceutical composition.

The compositions according to the invention may contain any suitable solvent and/or any suitable vehicle and/or any suitable excipient, optionally in combination with other compounds of interest.

As a result, for these compositions, the excipient contains, for example, at least one compound chosen from the group comprising preserving agents, emollients, emulsifiers, surfactants, moisturizers, thickeners, conditioners, mattifying agents, stabilizers, antioxidants, texturing agents, gloss agents, film-forming agents, solubilizers, pigments, colorants, fragrances and sunscreens. These excipients are preferably chosen from the group consisting of amino acids and derivatives thereof, polyglycerols, esters, cellulose polymers and derivatives, lanolin derivatives, phospholipids, lactoferrins, lactoperoxidases, sucrose-based stabilizers, vitamins E and derivatives thereof, natural and synthetic waxes, plant oils, triglycerides, unsaponifiable matter, phytosterols, plant esters, silicones and derivatives thereof, protein hydrolyzates, jojoba oil and derivatives thereof, liposoluble/water-soluble esters, betaines, amine oxides, plant extracts, sucrose esters, titanium dioxides, glycines, and parabens, and more preferably from the group consisting of butylene glycol, steareth-2, steareth-21, glycol-15 steelyl ether, cetearyl alcohol, phenoxyethanol, methylparaben, ethylparaben, propylparaben, butylparaben, butylene glycol, natural tocopherols, glycerol, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, glycerol, bisabolol, a dimethicone, sodium hydroxide, PEG-30 dipolyhydroxystearate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8, beeswax, glycerides from hydrogenated palm kernel oil, glycerides from hydrogenated palm oil, lanolin oil, sesame oil, cetyl lactate, lanolin alcohol, castor oil, titanium dioxide, lactose, sucrose, low-density polyethylene, an isotonic saline solution, and mixtures thereof.

Many cosmetically active ingredients are known to those skilled in the art for improving the health and/or the physical appearance of the skin (including the scalp) and/or mucous membranes. A person skilled in the art knows how to formulate cosmetic, nutraceutical or dermatological compositions to obtain the best effects. Moreover, these compounds may have a synergistic effect when they are combined with each other. These combinations are also covered by the present invention. The CTFA Cosmetic Ingredient Handbook, Second Edition (1992) describes various cosmetic and pharmaceutical ingredients commonly used in the cosmetics and pharmaceutical industry, which are suitable in particular for topical use. Examples of these classes of ingredients comprise, without being limited thereto, the following compounds: abrasives, absorbents, compounds for esthetic purposes such as fragrances, pigments, dyes, essential oils, astringents, etc. (for example clove oil, menthol, camphor, eucalyptus oil, eugenol, menthyl lactate, witch hazel distillate), antiacne agents, anti-flocculant agents, antifoam agents, antimicrobial agents (for example: iodopropyl butylcarbamate), antioxidants, binders, biological additives, buffer agents, swelling agents, chelating agents, additives, biocidel agents, denaturing agents, thickeners, and vitamins, and derivatives or equivalents thereof, film-forming materials, polymers, opacifiers, pH adjusters, reducing agents, depigmenting or lightening agents (for example: hydroquinone, kojic acid, ascorbic acid, magnesium ascorbyl phosphate, ascorbyl glucosamine), conditioning agents (for example: humectants).

The cosmetic composition may moreover comprise other cosmetic and/or nutraceutical agents having the same properties and inducing a synergistic or non-synergistic effect with the strain of Lactobacillus crispatus according to the invention, or cosmetic agents with complementary effects.

These may be, for example, antiwrinkle ingredients, agents stimulating the activity or proliferation of fibroblasts, agents stimulating the molecules of the extracellular matrix.

They may moreover be antiaging active ingredients and/or tensioning agents for a synergistic effect with the Lactobacillus crispatus strain. Advantageously, they are active ingredients which increase the gene and/or protein expression of collagen or active ingredients which prevent the degradation of collagen, such as retinol, vitamin C, an extract of Davilla rugosa sold under the name Collguard™ by BASF Beauty Care Solutions, a Hibiscus abelmoschus seed extract sold under the name Linefactor™, a peptide sold under the name Dermican™ by the Applicant, or products sold under the names Matrixyl™, Matrixyl 3000™ and Regestril™ by the company Sederma or Neuroguard by the company Codif, or Eternaline™ by the company Silab.

The tensioning agents that may be used in the invention may be chosen from synthetic polymers, such as polyurethane latexes or acrylic latexes, polymers of natural origin, notably polyholosides in the form of starch or in the form of carrageenans, alginates, agars, gellans, cellulose-based polymers and pectins; plant proteins and protein hydrolyzates; mixed silicates; wax microparticles; colloidal particles of inorganic filler chosen, for example, from silica, silica-alumina composites; and mixtures thereof.

Finally, they may be cosmetic ingredients, for instance antimicrobial agents, free-radical scavengers, soothing, calming or relaxing agents, agents acting on the microcirculation to improve the radiance of the complexion, in particular of the face, healing agents or slimming agents. Advantageously, the cosmetic and/or dermatological composition of the present invention also contains one or more tensioning agents and/or one or more antimicrobial agents and/or one or more free-radical scavengers and/or one or more soothing agents and/or one or more slimming agents and/or one or more agents acting on the microcirculation.

Among the antimicrobial agents combined with the Lactobacillus crispatus strain in a preferential mode of the present invention, mention may be made of 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, octoxyglycerol, octanoylglycine, caprylyl glycol, 10-hydroxy-2-decanoic acid, farnesol, phytosphingosines and mixtures thereof.

The free-radical scavengers may be vitamin C and derivatives thereof including ascorbyl glucoside, phenols and polyphenols, in particular tannins, ellagic acid and tannic acid; epigallocatechin and natural extracts containing it, in particular green tea extracts; anthocyanins; phenolic acids, stilbenes; monocyclic or polycyclic aromatic compound scavengers, tannins such as ellagic acid and indole derivatives and/or heavy metal scavengers such as EDTA, free-radical scavengers such as vitamin E and derivatives thereof such as tocopheryl acetate; bioflavonoids; coenzyme Q10 or ubiquinone.

As calmatives included in the composition of the invention, use may be made of pentacyclic triterpenes, ursolic acid and salts thereof, oleanolic acid and salts thereof, betulinic acid and salts thereof, salts of salicylic acid and in particular zinc salicylate, bisabolol, allantoin, omega-3 unsaturated oils, cortisone, hydrocortisone, indomethacin and betamethasone, antiinflammatory active agents, and notably those described in patent application FR 2 847 267, in particular the root extract of Pueraria lobata sold under the name Inhipase™ by the Applicant, and extracts of Theobroma cacao.

The active ingredients acting on the microcirculation, vasoprotectors or vasodilators, may be chosen from flavonoids, ruscogenins, nicotinates and essential oils.

The slimming active agents may notably be chosen from lipoprotein lipase inhibitors such as those described in patent US 2003/086949 (Coletica) and in particular an extract of cat's claw (Uncaria tornentosa), draining actives, notably hesperitin laurate (Flavagrum™), or quercitin caprylate (Flavenger™); phosphodiesterase enzyme inhibitors, adenylate cyclase activators, cAMP and/or active agents that are capable of scavenging spermine and/or spermidine. Mention may be made of an extract of Coleus forskohlii root, an extract of Cecropia obtusa, Uva lactuca, caffeine, forskolin, theophylline, theobromine and/or derivatives thereof, a hydrolyzed kappa carrageenan product named Slimexcess™ sold by the Applicant and/or mixtures thereof.

A subject of the present invention is also a cosmetic care process comprising the topical application to at least one area of healthy skin and/or healthy mucous membranes of a strain of Lactobacillus crispatus according to the invention, or of a cosmetic composition comprising it, for maintaining and/or increasing the expression of extracellular matrix molecules of healthy skin and/or healthy mucous membranes, preferably for maintaining and/or increasing the expression of collagens of healthy skin and/or healthy mucous membranes and/or elastic fibers of healthy skin and/or healthy mucous membranes, and/or for maintaining and/or increasing the biomechanical properties of healthy skin and/or healthy mucous membranes, notably the firmness and/or elasticity and/or density, preferably the firmness.

Advantageously, the strain of Lactobacillus crispatus according to the invention, preferentially in the form of a cosmetic composition according to the invention, is used in regular topical application and preferentially at least once a day, advantageously twice a day, for at least 10 days, preferentially for 20 days, and more preferentially for at least 28 days.

Advantageously, the topical application of a strain of Lactobacillus crispatus according to the invention or of a cosmetic composition comprising it is performed on all or part of the body and/or face and/or scalp, preferentially the legs, thighs, arms, stomach, neckline, neck, armpits or lips, more preferentially all or part of the face, and preferentially the cheeks, forehead, chin, lips or contour of the eyes.

The cosmetic care process according to the invention for maintaining and/or increasing the expression of extracellular matrix molecules of healthy skin and/or healthy mucous membranes, preferentially for maintaining and/or increasing the expression of collagens of healthy skin and/or healthy mucous membranes and/or elastic fibers of healthy skin and/or healthy mucous membranes, and/or for maintaining and/or increasing the biomechanical properties of healthy skin and/or healthy mucous membranes, notably the firmness and/or elasticity and/or density, preferentially the firmness, advantageously comprises the following steps:
the identification on the individual of an area of healthy skin and/or healthy mucous membrane, for which it is desired to maintain and/or increase the expression of the extracellular matrix molecules of healthy skin and/or healthy mucous membrane, preferentially maintain and/or increase the expression of collagens of healthy skin and/or healthy mucous membranes and/or elastic fibers of healthy skin and/or healthy mucous membranes, and/or maintain and/or increase the biomechanical properties of healthy skin and/or healthy mucous membranes, notably the firmness and/or elasticity and/or density, preferentially the firmness, and
topical application to this area of healthy skin and/or healthy mucous membranes of a cosmetic composition comprising the strain of Lactobacillus crispatus according to the invention, in an amount that is effective for maintaining and/or increasing the expression of the extracellular matrix molecules of the healthy skin and/or healthy mucous membranes, preferentially for maintaining and/or increasing the expression of collagens of healthy skin and/or healthy mucous membranes and/or elastic fibers of healthy skin and/or healthy mucous membranes, and/or for maintaining and/or increasing the biomechanical properties of healthy skin and/or healthy mucous membranes, notably the firmness and/or elasticity and/or density, preferentially the firmness, namely in a content of Lactobacillus crispatus strain of:
between 1×10⁻⁴% and 10% (v/v) by volume, preferentially between 1×10⁻⁴% and 5% (v/v) by volume, more advantageously between 1×10⁻³% and 3% (v/v) by volume, more preferentially between 0.1% and 2% (v/v) by volume, relative to the total volume of the composition, and/or
between 1×10⁻⁴% and 10% (w/w) by weight, preferentially between 1×10⁻⁴% and 5% (w/w) by weight, more advantageously between 1×10⁻³% and 0.5% (w/w) by weight, relative to the total weight of the composition.

For the purposes of the present invention, the term “topical route” means the direct local application and/or spraying of the Lactobacillus crispatus strain or the composition according to the invention onto the surface of the area of healthy skin and/or healthy mucous membranes.

For the purposes of the present invention, the term “topically and/or orally acceptable” means an ingredient which is suitable for topical and/or oral application, respectively, which is nontoxic, non-irritant to the skin including the scalp and which does not induce an allergic response, and which is not chemically unstable.

A subject of the invention is also the strain of Lactobacillus crispatus according to the invention, alone or in a dermatological composition comprising it, for its use, advantageously via the topical route, for treating and/or preventing pathologies of the skin and/or mucous membranes involving a decrease in the amount and/or expression of extracellular matrix molecules of the skin and/or mucous membranes and/or a decrease in the amount and/or expression of collagens and/or elastic fibers of the skin and/or mucous membranes, and/or a decrease in the amount and/or biomechanical properties of the skin and/or mucous membranes, notably a decrease in the firmness of the skin and/or mucous membranes and/or a decrease in the elasticity of the skin and/or mucous membranes, and/or a decrease in the density of the skin and/or mucous membranes.

For the purposes of the present invention, from a dermatological point of view, the term “treating and/or preventing skin and/or mucous membrane pathologies involving a decrease in the firmness of the skin and/or mucous membranes” means an increase, for therapeutic purposes, in the firmness of skin and/or mucous membranes which have lost firmness as a result of skin and/or mucous membrane pathologies, for instance rosacea or telangiectasia.

For the purposes of the present invention, from a dermatological point of view, the term “treating and/or preventing skin and/or mucous membrane pathologies involving a decrease in skin and/or mucous membrane elasticity” means an increase, for therapeutic purposes, in the elasticity of skin and/or mucous membranes which have lost their elasticity as a result of skin and/or mucous membrane pathologies, for instance solar elastosis and/or cutis laxa pathology.

For the purposes of the present invention, from a dermatological point of view, the term “treating and/or preventing skin and/or mucous membrane diseases involving a decrease in skin and/or mucous membrane density” means an increase, for therapeutic purposes, in the density of skin and/or mucous membranes which have lost their density as a result of skin and/or mucous membrane diseases such as Ehlers-Danlos syndrome and/or dermatoporosis.

The strain of Lactobacillus crispatus according to the invention may be in the form of a pharmaceutical composition, preferentially a dermatological composition, comprising at least one pharmaceutically and/or dermatologically acceptable excipient. In one embodiment of the invention, said composition is applied topically and/or orally, preferentially topically.

Advantageously, it is present in the pharmaceutical, preferentially dermatological, composition at a concentration of 1×10⁻⁴% to 10% by weight, preferentially between 1×10⁻⁴% and 5% by weight, more advantageously between 1×10⁻³% and 0.5% by weight relative to the total weight of the composition, said composition also comprising at least one pharmaceutically acceptable excipient.

Advantageously, the strain of Lactobacillus crispatus according to the invention is present in the pharmaceutical, preferentially dermatological, composition at a concentration of 1×10⁻⁴% to 10% (v/v) by volume, preferentially of 1×10⁻⁴% to 5% (v/v) by volume, more advantageously from 1×10⁻³% to 0.5% (v/v) by volume, more preferentially between 0.1% and 2% (v/v) by volume, relative to the total volume of the composition, said composition also comprising at least one pharmaceutically acceptable excipient.

The invention will be better understood on reading the description of the figures and examples that follow.

Examples referring to the description of the invention are presented below. These examples are given for illustrative purposes and shall in no way limit the scope of the invention. Each of the examples has a general scope.

The examples form an integral part of the present invention, and any feature appearing to be novel over any prior art whatsoever, from the description taken in its entirety, including the examples, forms an integral part of the invention.

Moreover, in the examples, all the percentages are given on a weight basis, unless otherwise indicated, the temperature is expressed in degrees Celsius, unless otherwise indicated, and the pressure is atmospheric pressure, unless otherwise indicated.

### EXAMPLE 1. PREPARATION OF DIFFERENT FORMS OF LACTOBACILLUS CRISPATUS

### Example 1.a: Production of Viable Whole Form of L. Crispatus

Isolated L. crispatus bacteria (CNCM I-5579) were seeded in MRS culture medium, incubated at 37° C. anaerobically. The pH was maintained at a value of pH=6, and the medium was incubated for 24 h. At the end of incubation, the medium was centrifuged, the cell concentrate was resuspended in the fermentation medium and freeze-dried in the presence of maltodextrin, for a final maltodextrin amount of 70% (w/w) by weight relative to the total weight of the ingredient.

### Example 1.b: Production of the Inactivated Whole Form of L. crispatus

Isolated L. crispatus bacteria (CNCM I-5579) were seeded in MRS culture medium, incubated at 37° C. anaerobically. The pH was maintained at a value of pH=6, and the medium was incubated for 24 h. At the end of the incubation, the medium was centrifuged and the cell concentrate was inactivated by heating it at 80° C. for 30 minutes. The cell concentrate was then resuspended in glycerol.

### Example 1.c: Production of L. crispatus Bacterial Lyzate

Isolated L. crispatus bacteria (CNCM I-5579) were seeded in MRS culture medium, incubated at 37° C. anaerobically. The pH was maintained at a value of pH=6, and the medium was incubated for 24 h. At the end of the incubation, the whole was treated in a high-pressure homogenizer. The lyzate was then resuspended in glycerol.

### Example 1.d: Production of the L. crispatus Bacterial Secretome

Isolated L. crispatus bacteria (CNCM I-5579) were seeded in MRS culture medium, incubated at 37° C. anaerobically. The pH was maintained at a value of pH=6, and the medium was incubated for 24 h. At the end of incubation, the medium was filtered to recover the bacterial secretome. The secretome was then resuspended in glycerol.

### Example 1.e: Production of the L. crispatus Bacterial Secretome in Powder Form

Isolated L. crispatus bacteria (CNCM I-5579) were seeded in MRS culture medium, incubated at 37° C. anaerobically. The pH was maintained at a value of pH=6, and the medium was incubated for 24 h. At the end of incubation, the medium was filtered to recover the bacterial secretome. The secretome was then sprayed with maltodextrin, for a final maltodextrin amount of 90% (w/w) relative to the total weight of the ingredient.

### Example 1.f: Production of Viable Whole Form of L. crispatus

Isolated L. crispatus bacteria (CNCM I-5579) were seeded in MRS culture medium, incubated at 37° C. anaerobically. The pH was maintained at a value of pH=6, and the medium was incubated for 24 h. At the end of incubation, the medium was centrifuged, the cell concentrate was resuspended in the fermentation medium and freeze-dried in the presence of maltodextrin, for a final maltodextrin amount of 50% (w/w) by weight relative to the total weight of the ingredient.

### EXAMPLE 2: INCREASE IN THE EXPRESSION OF TYPE I COLLAGEN BY FIBROBLASTS IN THE PRESENCE OF THE L. CRISPATUS STRAIN ACCORDING TO THE INVENTION

“Normal” human fibroblasts, i.e. free of pathology, obtained from a healthy 34-year-old donor were seeded in 24-well plates and then cultured in DMEM medium until confluent.

Incubation was continued for 48 hours in the presence of L. crispatus bacteria according to Example 1.a seeded in gondolas above the culture wells in MRS medium diluted with PBS to a concentration of 10⁶ CFU/m L. The same culture medium without addition of the strain according to the invention was used as a control (Control).

At the end of incubation, the bacteria were removed and the cells were lyzed with ammonium hydroxide buffer. The buffer was then removed, and the wells were saturated with a mixture of PBS/BSA (Phosphate-Buffered Saline/Bovine Serum Albumin).

After saturation, anti-collagen I primary antibody (Interchim/Acris, Montluçcon, France, LH0284/R1038) was incubated for a few minutes, followed by Europium-conjugated secondary antibody (Perkin Elmer) and the fluorescence is then measured at (λexc. 340 nm/λem. 615 nm) using an EnVision® multi-label plate reader (Perkin Elmer). The fluorescence results were normalized relative to the fluorescence obtained with the same cell medium in the absence of the L. crispatus strain according to the invention (Control) and were related to the amount of DNA obtained under each condition. The results presented correspond to the mean of 6 assays (n=6).

Results

| | | L. crispatus |
|---|---|---|
| | Control | strain according to |
| | (culture medium) | Example 1.a |
| Stimulation | 100 | 165 |
| average (%) | | |
| Standard deviation | 26 | 32 |
| Statistics/Control | — | (*) p < 0.05 |
| (one-way ANOVA) | | |

The L. crispatus strain according to Example 1.a, product of the invention, showed significant stimulation of collagen I by human skin fibroblasts cultured in vitro. It can thus be used to increase the firmness of healthy skin and/or healthy mucous membranes.

### EXAMPLE 3: INCREASE IN THE EXPRESSION OF THE PROTEINS FIBULIN-5 AND EMILIN-1 INVOLVED IN THE FORMATION OF ELASTIC FIBERS BY FIBROBLASTS IN THE PRESENCE OF THE STRAIN OF L. CRISPATUS ACCORDING TO THE INVENTION

“Normal” human fibroblasts, i.e. free of pathology, obtained from a 19-year-old healthy donor were cultured in FGM (Fibroblast Growth Medium). Once cultured, incubation was continued for 48 hours in the presence of the L. crispatus bacterial secretome obtained according to Example 1.e at a concentration of 0.1% (w/w) relative to the total weight of the culture medium and of the secretome. The same culture medium without addition of the strain according to the invention was used as a control (Control).

At the end of incubation, the culture medium was removed and the cells were lyzed with lysis buffer.

Fibulin-5 and emilin-1 were identified and quantified by means of a capillary electrophoresis-based protein analysis system. An amount of protein obtained from the cell lyzates, namely 0.2 mg/mL of lyzate for fibulin-5 and 0.2 mg/mL of lyzate supernatant for emilin-1, was deposited in capillaries to which a primary anti-fibulin-5 or anti-emilin-1 antibody was added followed by a secondary antibody conjugated to horseradish peroxidase and a chemiluminescent substrate. The whole was incubated for 30 minutes, and the chemiluminescent signal resulting from the detection of fibulin-5 protein was then quantified by capillary electrophoresis using suitable software.

The results are the mean of the assays performed ±standard deviation, expressed as a percentage, relative to the untreated control normalized to 100%. The statistical analysis of the results was done relative to the untreated control using the One-Way ANOVA test. The significance level was set at 5%.

Results

| | | L. crispatus secretome |
|---|---|---|
| | Control | according to Example 1.e |
| | (Culture medium) | at 0.1% (w/w) |
| Fibulin-5 percentage | 100 | 150 |
| stimulation average | | |
| Standard deviation | 8.2 | 16.6 |
| Statistics | NA | P < 0.001 |

The secretome of the Lactobacillus crispatus strain according to Example 1.e of the invention showed a significant 50% increase in fibulin-5 protein expression by human skin fibroblasts cultured in vitro. Thus, the Lactobacillus crispatus strain, and in particular its secretome, can be used to increase the elasticity of healthy skin and/or healthy mucous membranes.

| | | L. crispatus secretome |
|---|---|---|
| | Control | according to Example 1.e |
| | (Culture medium) | at 0.1% (w/w) |
| Emilin-1 percentage | 100 | 153.1 |
| stimulation average | | |
| Standard deviation | 12.7 | 15.1 |
| Statistics | NA | P < 0.001 |

The secretome of the Lactobacillus crispatus strain according to Example 1.e of the invention showed a significant 53% increase in protein expression of emilin-1 by human skin fibroblasts cultured in vitro. Thus, the Lactobacillus crispatus strain, and in particular its secretome, can be used to increase the elasticity of healthy skin and/or healthy mucous membranes.

### EXAMPLE 4: IN VIVO MEASUREMENT OF THE INCREASE IN SKIN ELASTICITY IN THE PRESENCE OF THE STRAIN OF L. CRISPATUS ACCORDING TO THE INVENTION

A population of 30 women from 40 to 50 years old showing a decrease in skin elasticity on the face applied to one half of the face a cream in the form of an emulsion as described in Example 7b), comprising a final weight concentration of the secretome of the L. crispatus strain according to Example 1.e of 1% (w/w) relative to the total weight of the cream or in the form of the same “placebo” emulsion in which said secretome was replaced with water, at a rate of two applications per day for 2 months.

The evaluation of the biomechanical properties of the skin was performed using a Cutometer®. This clinical evaluation equipment has the capacity to measure three different parameters, namely the overall elasticity, the immediate elasticity, and the elastic recovery of the skin, all of which reflect the elasticity of the skin. From these measurements, different parameters reflecting the state of elasticity of the skin were deduced.

The overall and immediate elasticity and also the elastic recovery of the skin were measured with a Cutometer® at the start of the study (DO), after one month of application (D28) and finally after 2 months of application (D56).

The efficacy of the secretome of the Lactobacillus crispatus strain according to Example 1.e was compared with that obtained with the placebo emulsion.

Results

| | | | Formulation containing the | |
|---|---|---|---|---|
| | Placebo | | L. crispatus strain according | |
| | formulation | | to Example 1.e at 0.1% (w/w) | |
| | At D28 | At D56 | At D28 | At D56 |
| Percentage | 1.1% | 2.7% | +4.4% (p < 0.001 | +12% p < 0.001 |
| improvement | | | vs D0) and | vs D0 and |
| in the overall | | | p < 0.05 vs | vs placebo) |
| elasticity | | | placebo | |
| Percentage | 5.9% | 1.6% | 5.8% (p < 0.05 | 11.7% (p < 0.001 |
| improvement | (p < 0.05 | | vs D0) | vs D0 and |
| in the net | vs D0) | | | vs placebo) |
| elasticity | | | | |
| Percentage | 2.9% | 2.7% | 4.7% (p < 0.001 | 12.5% (p < 0.001 |
| improvement | | | vs D0) | vs D0 and |
| in the elastic | | | | vs placebo) |
| recovery | | | | |

After 2 months of use, the formulation containing the secretome of the Lactobacillus crispatus strain according to Example 1.e induced a significant increase in all the elasticity parameters. This increase is significant relative to the start of the study at DO, but also relative to the placebo.

These results show the beneficial effect of the formulated Lactobacillus crispatus strain, in particular its secretome, for maintaining and/or increasing in vivo the biomechanical properties of healthy skin and/or healthy mucous membranes, in particular the elasticity of healthy skin and/or healthy mucous membranes.

### EXAMPLE 5: IN VIVO MEASUREMENT OF THE INCREASE IN SKIN DENSITY IN THE PRESENCE OF THE STRAIN OF L. CRISPATUS ACCORDING TO THE INVENTION

A population of 31 women from 45 to 65 years old showing wrinkles on the forehead and a decrease in the biomechanical properties of the skin on the face applied to one half of the face a cream in the form of an emulsion as described in the formulation presented in Example 7.c, comprising a final weight concentration of the L. crispatus strain obtained according to Example 1.f of 0.05% (w/w) relative to the total weight of the cream or in the form of the same “placebo” emulsion in which said strain was replaced with water, at a rate of two applications per day for 2 months.

The evaluation of the skin density was performed using a scanner equipped with an ultrasound probe. The scanner allows visualization of the skin density, which is a reflection of the synthesis of extracellular matrix compounds.

The improvement in skin density and thickness was measured at the start of the study (D0), after one month of application (D28) and finally after 2 months of application (D56).

The efficacy of the Lactobacillus crispatus strain according to Example 1.f was compared with that of a placebo emulsion.

Results

| | | Formulation containing the L. |
|---|---|---|
| | Placebo | crispatus strain according to |
| | formulation | Example 1.f at 0.05% (w/w) |
| | At D56 | At D56 |
| Percentage increase in | Not significant | +6% (p < 0.05 vs D0 and vs |
| dermal density vs D0 | | placebo) |

After 2 months of use, the formulation of the Lactobacillus crispatus strain according to Example 1.f induced a significant increase in skin density. This increase is significant relative to the start of the study at DO, but also relative to the placebo.

These results show the beneficial effect of the formulated Lactobacillus crispatus strain for maintaining and/or increasing in vivo the biomechanical properties of healthy skin and/or healthy mucous membranes, in particular the density of healthy skin and/or healthy mucous membranes.

### EXAMPLE 6: EXAMPLE OF A COSMETIC INGREDIENT COMPRISING THE LACTOBACILLUS CRISPATUS STRAIN ACCORDING TO THE INVENTION

| | |
|---|---|
| Lactobacillus crispatus extract | 0.2% |
| according to Example 1.c | |
| Glycerol | 80% |
| Water | qs 100 |

### EXAMPLE 7: EXAMPLE OF A COSMETIC COMPOSITION COMPRISING THE STRAIN OF LACTOBACILLUS CRISPATUS ACCORDING TO THE INVENTION

### Example 7.a: Example of a Cosmetic Composition Comprising the Lactobacillus Crispatus Strain According to Example 1.c

Methods known to those skilled in the art are used to mix together the various phases A, B, C and D below to prepare a composition according to the present invention. The proportions are expressed as percentages and the names in capital letters correspond to the INCI names of the ingredients.

Phase A

| | |
|---|---|
| GLYCERYL STEARATE AND PEG-100 STEARATE | 4.00 |
| PENTAERYTHRITYL DISTEARATE | 1.50 |
| CETEARYL ISONONANOATE | 3.00 |
| PROPYLHEPTYL CAPRYLATE | 5.00 |
| COCOYL CAPRYLATE | 2.00 |
| DICAPRYLYL CARBONATE | 3.00 |
| DIMETHICONE | 1.00 |

Phase B

| | |
|---|---|
| Water | 64.43 |
| Propylene glycol, phenoxyethanol, | 1.00 |
| chlorphenesin, methylparaben | |
| Glycerol | 1.57 |
| Xanthan gum | 0.20 |
| Butylene glycol | 2.00 |
| Sodium hydroxide, water | 0.15 |

Phase C

| | |
|---|---|
| Carbomer | 0.15 |
| Water | 10 |

Phase D

| | |
|---|---|
| Extract of Lactobacillus crispatus obtained | 1.00 |
| according to Example 1c) | |

### Example 7.b: Example of a Cosmetic Composition Comprising the Lactobacillus Crispatus Strain According to Example 1.e

The proportions are expressed as percentages and the names in capital letters correspond to the INCI names of the ingredients.

Phase A

| | |
|---|---|
| GLYCERIN | 5.00 |
| BUTYLENE GLYCOL | 10.00 |
| XANTHAN GUM | 2.00 |

Phase B

| | |
|---|---|
| Water | 58.95 |
| Sodium benzoate | 0.25 |
| GLYCERINE, WATER, SODIUM | 1.00 |
| LEVULINATE, SODIUM ANISATE | |

Phase C

| | |
|---|---|
| POLYGLYCERYL-2 DIPOLYHYDROXYSTEARATE | 1.00 |
| DIPROPYLHEPTYL CARBONATE | 3.00 |
| DICAPRYLYL CARBONATE | 3.00 |
| CAPRYLYL CAPRYLATE/CAPRATE | 10.00 |
| LAURETH-7 CITRATE | 0.50 |

Phase D

| | |
|---|---|
| Citric acid | 0.30 |

Phase E

| | |
|---|---|
| Extract of Lactobacillus crispatus | 1.00 |
| obtained according to Example 1e | |
| Water | 4.00 |

Methods known to those skilled in the art are used to mix together the various phases A, B and C at room temperature, by adding B to A with stirring, followed by adding C to AB. The pH is adjusted to 4.9 with D. Then phase E is added. Finally, homogenization is performed with an Ultra-Turrax blender to prepare a composition according to the present invention.

### Example 7.c: Example of a Cosmetic Composition Comprising the Lactobacillus Crispatus Strain According to Example 1.f

The Lactobacillus crispatus strain obtained according to Example 1.f is added just before application to the skin in a formulation as defined below according to Table 8, at a final concentration of 0.05% (w/w) relative to the total weight of the formulation.

| | Amount |
|---|---|
| Name | (% by total weight) |
| Phase A | |
| Cetearyl alcohol, lecithin, sodium | 3.00 |
| cetearyl sulfate, olus oil | |
| Hydrogenated plant glycerides | 2.50 |
| Caprylyl caprylate/caprate | 6.50 |
| Dicaprylyl carbonate | 4.00 |
| Phase B | |
| Water | 66.35 |
| Butylene glycol | 10.00 |
| Sodium benzoate | 0.25 |
| Phase C | |
| Glycerin | 5.00 |
| Xanthan gum | 1.00 |
| Phase D | |
| Glycerol, aqua, sodium levulinate, | 1.00 |
| sodium anisate | |
| Citric acid | 0.40 |

### EXAMPLE 8: EXAMPLE OF A DERMATOLOGICAL COMPOSITION

### Example 8a): Ointment Containing the Viable Whole Strain of L. crispatus According to the Invention

The composition below is prepared according to methods known to those skilled in the art, in particular as regards the various phases to be mixed together.

The amounts indicated are as weight percentages relative to the total weight of the composition.

| | |
|---|---|
| Ingredient * | 3.00 |
| Excipient: | |
| Low density polyethylene | 5.50 |
| Liquid petroleum jelly | qs 100 |

* The ingredient is prepared according to the preceding Example 1a). The ingredient according to the invention is sterilized and then dried before being incorporated into the composition in the form of an ointment.

### Example 8b): Cream Containing the Strain of Lactobacillus crispatus According to Example 1c

| | | Amount |
|---|---|---|
| | | (% by total |
| Phase | Name | weight) |
| A | Water | 79.60 |
| A | PROPYLENE GLYCOL (AND) | 2.50 |
| | PHENOXYETHANOL (AND) | |
| | CHLORPHENESIN | |
| | (AND) METHYLPARABEN | |
| A | Glycerol | 2 |
| A | SODIUM STEAROYL GLUTAMATE | 0.5 |
| B | DICAPRYLYL CARBONATE | 3 |
| B | COCOYL CAPRYLATE/CAPRATE | 3 |
| B | CAPRYLIC/CAPRIC TRIGLYCERIDE | 3 |
| B | SUCROSE POLYSTEARATE | 3 |
| | (AND) CETYL PALMITATE | |
| B | PENTAERYTHRITYL DISTEARATE | 1 |
| B | SODIUM POLYACRYLATE | 0.7 |
| C | Water | 0.9 |
| C | Lactobacillus crispatus extract | 0.1 |
| | according to Example 1c) | |
| D | Citric acid and water | 0.7 |

Phases A and B are heated to 75° C. and then mixed with stirring. Once the mixture has returned to room temperature, phases C and D are added with stirring.

1.-16. (canceled)

17. A method for maintaining and/or increasing the expression of extracellular matrix molecules of healthy skin and or healthy mucous membranes comprising the cosmetic and/or nutraceutical use of the strain of Lactobacillus crispatus.

18. The method as claimed in claim 17, in which the extracellular matrix molecules of healthy skin and/or healthy mucous membranes are chosen from collagen fibers of healthy skin and/or healthy mucous membranes, and/or elastic fibers of healthy skin and/or healthy mucous membranes.

19. The method as claimed in claim 17, for maintaining and/or increasing the expression of type I, III, IV, V, VI, VII, XII, XIII, XIV, XVI, XVII, XXIV and/or XXIX collagens.

20. The method as claimed in claim 17, for maintaining and/or increasing the biomechanical properties of healthy skin and/or healthy mucous membranes.

21. The method as claimed in claim 17, in which the strain is used in whole form, notably viable and/or inactivated, notably dead, and/or in the form of a lyzate, and/or in the form of one or more of its fractions, and/or in the form of one or more of its metabolites.

22. The method as claimed in claim 17, in which the strain is used in viable whole form and/or in lyzate form and/or in which the strain used is the Lactobacillus crispatus strain deposited under the designation CNCM I-5579.

23. The method as claimed in claim 17, in which the Lactobacillus crispatus strain is combined with its fermentation and/or culture medium.

24. The method as claimed in claim 17, in which the Lactobacillus crispatus strain is applied topically to healthy skin and/or healthy mucous membranes.

25. The method as claimed in claim 17, such that the strain of Lactobacillus crispatus is present in the cosmetic composition at a concentration of 1×10⁻⁴% to 10% by weight, relative to the total weight of the composition, said composition also comprising at least one cosmetically acceptable excipient.

26. A cosmetic care process, characterized in that it comprises the topical application to at least one area of healthy skin and/or healthy mucous membranes of a strain of Lactobacillus crispatus in which the strain is used in whole form, notably viable and/or inactivated, notably dead, and/or in the form of a lyzate, and/or in the form of one or more of its fractions, and/or in the form of one or more of its metabolites, or of a cosmetic composition as claimed in claim 25, for maintaining and/or increasing the expression of extracellular matrix molecules of healthy skin and/or healthy mucous membranes, and/or for maintaining and/or increasing the biomechanical properties of healthy skin and/or healthy mucous membranes, notably the firmness and/or elasticity and/or density.

27. The cosmetic care process as claimed in claim 26, in which the topical application of a strain of Lactobacillus crispatus is performed on all or part of the body and/or face and/or scalp.

28. The cosmetic care process as claimed in claim 26, in which the cosmetic composition comprises the strain of Lactobacillus crispatus in which the strain is used in whole form, notably viable and/or inactivated, notably dead, and/or in the form of a lyzate, and/or in the form of one or more of its fractions, and/or in the form of one or more of its metabolites, relative to the total weight of the composition, said composition also comprising at least one cosmetically acceptable excipient.

29. A method comprising a strain of Lactobacillus crispatus, or a dermatological composition comprising it, for treating and/or preventing pathologies of the skin and/or mucous membranes involving a decrease in the amount and/or expression of extracellular matrix molecules of the skin and/or mucous membranes and/or a decrease in the amount and/or expression of collagens and/or elastic fibers of the skin and/or mucous membranes, and/or a decrease in the biomechanical properties of the skin and/or mucous membranes, notably a decrease in the firmness of the skin and/or mucous membranes and/or a decrease in the elasticity of the skin and/or mucous membranes, and/or a decrease in the density of the skin and/or mucous membranes, the strain being used in whole form, notably viable and/or inactivated, notably dead, and/or in the form of a lyzate, and/or in the form of one or more of its fractions, and/or in the form of one or more of its metabolites.

30. The method of claim 29, wherein the strain of Lactobacillus crispatus is in the form of a dermatological composition comprising at least one dermatologically acceptable excipient.

31. The method of claim 29, wherein the strain of Lactobacillus crispatus is characterized in that it is present in the pharmaceutical composition at a concentration of 1×10⁻⁴% to 10% by weight, relative to the total weight of the composition, said composition also comprising at least one pharmaceutically acceptable excipient.

32. The method of claim 29, wherein the strain of Lactobacillus crispatus is characterized in that the strain is used in whole form, notably viable and/or inactivated, notably dead, and/or in the form of a lyzate, and/or in the form of one or more of its fractions, and/or in the form of one or more of its metabolites.
